(19) United States
(12) Patent Application Publication
(10) Pub. No.: US 2008/0172085 A1
(43) Pub. Date: Jul. 17, 2008

(54) Elliptical Excision Clamp

(76) Inventors: Kwok Wai Chiu, Plano, TX (US); Timothy Chiu, Plano, TX (US); Kwok Hung Chung, Plano, TX (US)

Correspondence Address: Kwok Wai Chiu, 1309 Chesapeake Drive, Plano, TX, 75093, US

(21) Appl. No.: 11/623,412

(22) Filed: Jan. 16, 2007

(51) Int. Cl. A61B 17/28

(52) U.S. Cl. 606/205

(57) ABSTRACT

An elliptical excision clamp helps in the removal of superficial lesions and abnormalities as well as the subsequent suturing of said removal of lesions. An elliptical excision clamp utilizes a pair of half-elliptical shaped clamp jaws curved at an appropriate ratio for the target surgical area, a number of suture hole and slot pairs on the clamp jaws used to facilitate suturing, a crossed members array with finger loops, and a pivot point pin used to connect said crossed members. This device helps minimize bleeding and operation time, and maximizes surgical efficiency and wound management.

### BACKGROUND

1. Technical Field

The present invention relates to a skin excision device for use in surgically removing all or part of a skin (or dermal) lesion. More particularly, an enhanced skin excision device would allow for easier and more efficient skin lesion removal as well as minimized bleeding.

2. Description of Related Art

Skin excision is useful in fully removing skin cancers such as basal cell carcinoma, squamous cell carcinoma, and melanoma. Skin excision is also useful for lesion removal, infection management, and cosmetic purposes.

The most common form of skin excision is an elliptical excision. With an elliptical excision, the resulting scar runs parallel with existing skin creases and is relatively narrow and short. Ordinarily, a colored pen is used to demarcate the area to be removed. The surgeon will then use a scalpel and scissors to cut around and under the lesion until it is completely detached. There is sometimes a great deal of bleeding in the area where the lesion is removed. The bleeding may be checked by coagulating the blood vessels with surgical diathermy that accompanied with hissing sound and burning smell. After bleeding checked, the edges of the ellipse will then be sutured together in a thin line.

Owing to these complicated effects, a need exists for an improved surgical device for use in surgical procedures for removing superficial lesions via skin excision. Such a device should be simple and convenient to use, even by doctors with novice surgical skills, and allow for minimized side effects and provide faster healing procedures.

### SUMMARY OF THE INVENTION

Objects and advantages of the invention are set forth below.

It is a principle objective of the present invention to provide an elliptical skin excision clamp offering enhanced efficiency, simplicity, safety, ease of use, and reduced surgical time.

According to the present invention, an elliptical excision clamp utilizes a pair of clamp jaws measured at a variable, half-elliptical curve to accommodate the size of any target area, a crossed members array with finger loops, and a pivot point pin used to connect said crossed members. This device helps minimize bleeding and operation time, and maximizes surgical efficiency, healing, and recovery.

### BRIEF DESCRIPTION OF THE DRAWINGS

The novel features believed characteristic of the invention are set forth in the appended claims. The invention itself, however, as well as a preferred mode of use, further objectives and advantages thereof, will be best understood by reference to the following detailed description of illustrative embodiments when read in conjunction with the accompanying drawings, wherein:

FIG. 1 is a ¾ profile view of an elliptical excision device, as used during the skin excision procedure, in accordance with the present invention.

FIG. 2 is a side view of an elliptical excision device in accordance with the present invention.

FIG. 3 is a side view of a preferred embodiment comprising of three sets of angular facet 14, 15, and 16 appearing on the side of each clamping tab.

FIG. 4 is a ¾ profile view of an elliptical excision device in accordance with the present invention as it is being prepared to clamp around a skin lesion, which is demarcated by appropriate marker or pen.

FIG. 5 is a ¾ profile view of the elliptical excision device in use, clamping the skin surrounding a skin lesion.

FIG. 6 is a ¾ profile view of the elliptical excision device in use, clamping the skin surrounding a skin lesion, right before a scalpel is used to excise the skin lesion.

FIG. 7 is a ¾ profile view of the elliptical excision device in use, clamping the skin after the skin lesion has been removed and suturing can then be applied.

FIG. 8 is a top view of the sutured skin after the skin lesion has been removed.

### DETAILED DESCRIPTION

While the invention is described below with respect to a preferred embodiment, other embodiments are possible. The concepts disclosed herein apply equally to other instruments for removing samples of other tissues, organs, organic matter, and can be applied to inorganic matter as well, provided that they follow the spirit of the teachings disclosed herein.

Whereas many prior skin excision procedures simply make incisions around the skin lesion using a scalpel and scissors, the present invention enables one to remove the desired portion of the skin precisely with the use of a specialized elliptical excision clamp 101 that holds the specified area of skin around the lesion in place and subsequently allows for less bleeding and more efficient in suturing. Such efficient excision of the skin lesion is made possible with the present elliptical excision device as disclosed herein.

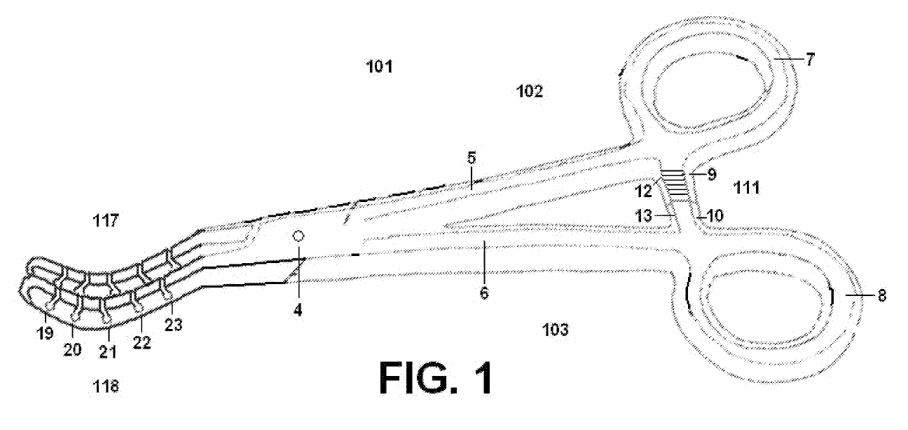

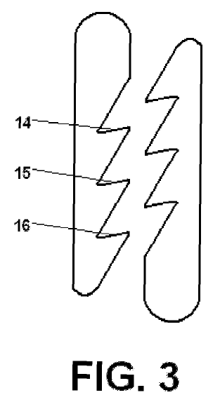

Referring to FIG. 1, the elliptical excision clamp is generally designated numeral 101. According to the present invention, the elliptical excision clamp 101 has: a pair of crossed members 102 and 103 and a pivot point 4 fixed between the crossed members. The pivot point 4 is defined as any means allowing for movement of crossed members 102 and 103 toward and away from each other about a common axis. The crossed members 102 and 103 comprise two handles 5 and 6 with finger loops 7 and 8 at the proximal end of crossed members 102 and 103. Clamping tabs 9 and 10 extend from each finger loop 7 and 8 in such a way that the clamping tabs 9 and 10 will overlap using the angular facets 12 and 13 as the handles 5 and 6 are closed 111. The clamping jaws 117 and 118 are located on the opposite end of the crossed members 102 and 103. Suture hole and slot pairs 19, 20, 21, 22, 23 are located on each clamping jaw 117 and 118. Referring to FIG. 3, the clamping teeth 14, 15, 16 are located on each of the angular facets 12 and 13 of clamping tabs 9 and 10.

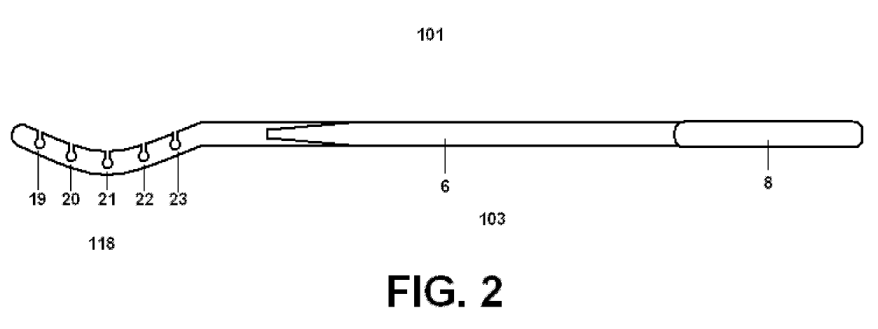

Referring to FIG. 2, a side view of the elliptical excision clamp 101 is shown. Most prominent is the clamp jaw 118 and the suture hole and slot pairs 19, 20, 21, 22, 23. Each suture hole leads to a slot opening on the upper margin of the clamp jaw. This is the key part of the invention, as it allows for the combined actions of clamping, cutting, and suturing in one motion. This also allows for minimal bleeding and a better looking wound.

In a preferred embodiment of the present invention, the dimensions of the elliptical excision clamp 101 are as follows. Referring to FIG. 1 the curvature of each of the clamping jaws 117 and 118 is a variable ratio, resembling a half ellipsis, determined by the situation and size of the target surgical area. The width and height of each of the clamping jaws 117 and 118 is also variable according to the need of the situation. The diameter and length of the suture hole and slot pairs 19, 20, 21, 22, 23 are likewise determined by need. Referring to FIG. 1, the length of each crossed member 102 and 103 is variable, determined by need.

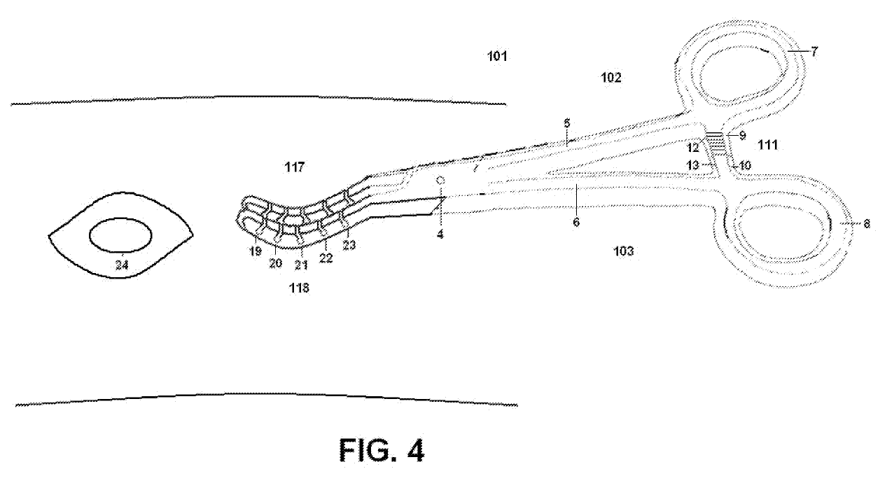

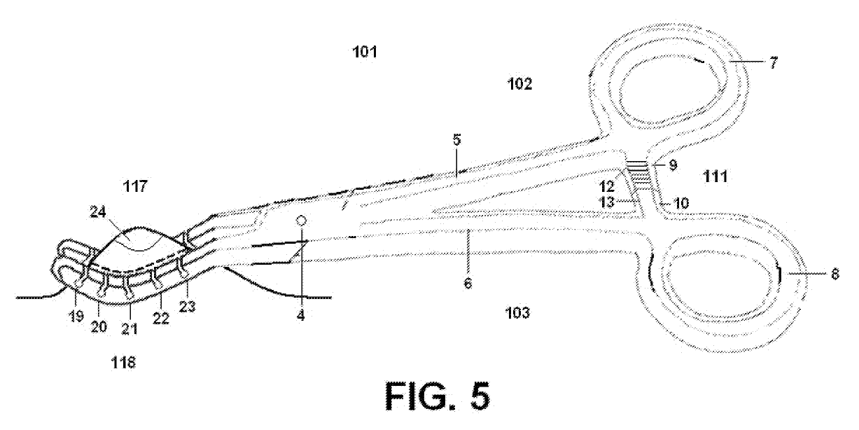

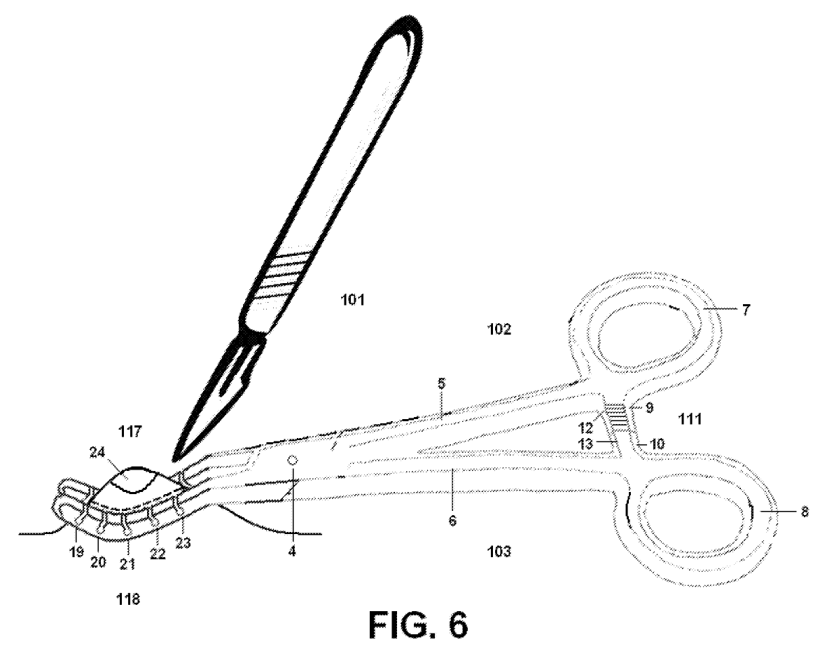

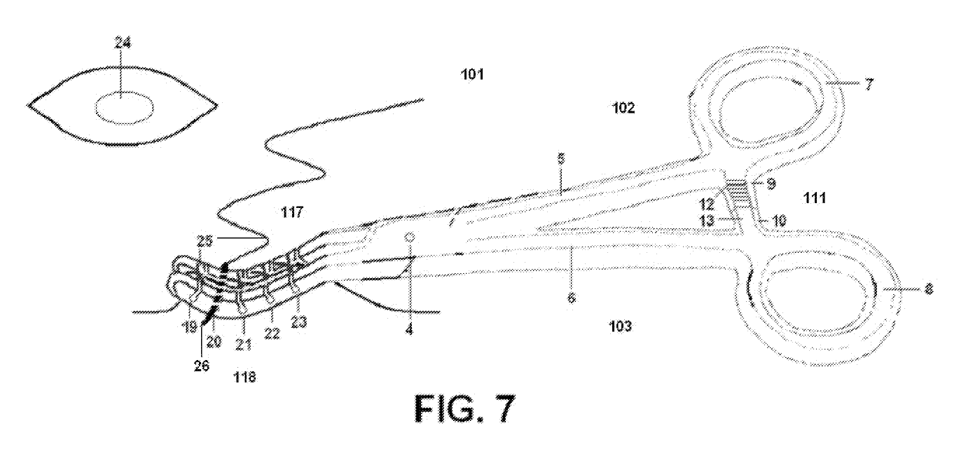

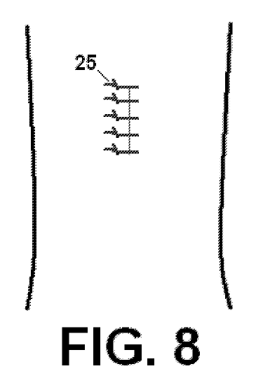

In practice, the selected area of skin is cleaned with appropriate sterilizing solution and anesthetized with appropriate medication. Referring to FIG. 4, the elliptical excision clamp 101 is prepared to use around the selected area of skin where the lesion is to be removed from. The area around the lesion on the targeted area of skin is demarcated with appropriate marker or pen. Referring to FIG. 5, the elliptical excision clamp 101 is used to clamp the demarcated area of skin around the lesion. Referring to FIG. 6, an appropriate surgical scalpel is used to remove the demarcated area of skin. Referring to FIG. 7, the elliptical excision clamp 101 is held in place, after the lesion 24 is removed. Suturing procedure can then be taken place by a suture threading apparatus, which comprises of needle 26 and suture material 25, through the elliptical excision clamp's 101 suture hole and slot pairs 19, 20, 21, 22, and 23. Referring to FIG. 8, suturing procedure is complete with suture material 25 and the elliptical excision clamp. Suture lines and knots are positioned on the operative skin wound.

The elliptical excision clamp 101 may be made of any material suitable for use in surgical procedures. In one embodiment of the present invention, the elliptical excision clamp 101 is made from stainless steel alloy. In another embodiment of the present invention, the elliptical excision clamp 101 is made of plastic materials and/or composite materials.

While the invention has been particularly shown and described with reference to a preferred embodiment, it will be understood by those skilled in the art that various changes in form and detail may be made therein without departing from the spirit and scope of the invention.

We claim:

1. An elliptical excision clamp for use in the removal of a lesion, said elliptical excision clamp comprising:
A pair of crossed members having proximal ends and distal ends; and
A pivot pin fixed between the crossed members allowing for movement of the crossed members toward and away from each other;
The crossed members comprising
Handles with finger loops at the proximal ends of the crossed members;
Clamping tabs extending from the finger loops wherein the clamping tabs will overlap when the handles are closed and wherein angular facets are formed on the clamping tabs such that the clamping tabs will lock together as the angular facts engage; and
Elliptical jaws on the distal ends of the crossed members having suture holes wherein suturing of dermal tissue can be facilitated.

2. An elliptical excision clamp according to claim wherein the elliptical excision clamp is used for skin excision and biopsy.

3. An elliptical excision clamp according to claim wherein the elliptical excision clamp is used for soft tissue excision and biopsy such as muscle excision and biopsy, breast tissue excision and biopsy, tongue lesion excision and biopsy or any other superficial lesion excision and biopsy.

4. An elliptical excision clamp according to claim 1 wherein the elliptical excision clamp is made of a stainless steel alloy.

5. An elliptical excision clamp according to claim 1 wherein the elliptical excision clamp is made of plastic materials and/or composite materials.
